(19) United States
(12) Patent Application Publication
(10) Pub. No.: US 2022/0133999 A1
(43) Pub. Date: May 5, 2022

(54) MONITORING OF PHYSIOLOGICAL PARAMETERS WITH IMPEDANCE MEASUREMENT

(71) Applicant: Medtronic, Inc., Minneapolis, MN (US)

(72) Inventors: Jason C. Lee, Edina, MN (US); Hyun J. Yoon, Vadnais Heights, MN (US); Jon E. Thissen, Rosemount, MN (US); Ashley L. Galarneau, Minneapolis, MN (US); Randal C. Schulhauser, Phoenix, AZ (US)

(21) Appl. No.: 17/085,963

(22) Filed: Oct. 30, 2020

(51) Int. Cl. A61M 5/172; G16H 40/63; G06N 20/00

(52) U.S. Cl. CPC A61M 5/1723; G16H 40/63; G06N 20/00; A61M 2230/65; A61M 2205/50; A61M 2230/06; A61M 5/142; A61M 2205/3303; A61M 2230/42; A61M 2205/3553; A61M 2205/502; A61M 2205/8206; A61M 2230/63; A61M 2230/201

(57) ABSTRACT

A system for monitoring a patient includes one or more processors and a sensor device implemented in circuitry. The system is configured to measure, using the sensor device, an impedance of tissue of the patient and determine, using one or more processors, a physiological parameter comprising at least one of a heart rate, cardiac output, vascular tone, perfusion level, fluid status, respiration effort, or respiration rate of the patient based on the impedance of the tissue of the patient. The system is configured to facilitate therapy, using the one or more processors, based on the determined physiological parameter.

### TECHNICAL FIELD

The disclosure relates to medical systems and, more particularly, to medical systems for monitoring physiological parameters of a patient for the treatment, management, and/or prevention of disease, including but not limited to, diabetes, heart failure, and/or neural disorders.

### BACKGROUND

A patient with diabetes receives insulin from a pump or injection device to control the glucose level in his or her bloodstream. Naturally produced insulin may not control the glucose level in the bloodstream of a diabetes patient due to insufficient production of insulin and/or due to insulin resistance. To control the glucose level, a patient's therapy routine may include dosages of basal insulin and bolus insulin. Basal insulin, also called background insulin, tends to keep blood glucose levels at consistent levels during periods of fasting and is a long acting or intermediate acting insulin. Bolus insulin may be taken specifically at or near mealtimes or other times where there may be a relatively fast change in glucose level, and may therefore serve as a short acting or rapid acting form of insulin dosage. Dosages of insulin may be informed, in some instances, by a glucose monitor, which may include, but is not limited to, a continuous glucose monitor (CGM). There exists a need specifically for a glucose monitor or CGM device that may leverage its communication with a patient to measure physiological parameters that may include blood glucose parameters, but also additional physiological parameters that may otherwise be indicative of the patient's ongoing health or physical condition on an ongoing basis without the need to attach or otherwise utilize secondary sensors beyond the blood glucose monitor or CGM.

### SUMMARY

Devices, systems, and techniques for managing glucose level and other physiological parameters in a patient are described. Medical devices (e.g., pump or injection device) may be in communication with, or otherwise use monitors having electrodes to perform various measurements for a patient. Such medical devices may include, for example, a percutaneous cannula configured to deliver insulin to the patient. The monitors having electrodes may concurrently monitor the patient's response to treatment introduced by such medical devices, however, monitors (including but not limited to a continuous glucose monitoring (CGM)), may be located on a patient's body at a position optimized for the treatment, management, and/or prevention of disease, but in a position that may not be immediately useful for measuring concurrent or secondary physiological parameters that may, nonetheless be useful for monitoring a patient's overall health and response to treatment. For example, a CGM device may be located far away from the heart (e.g., around a wrist, on an upper arm, above the chest, or in an abdominal region), which may attenuate electrical signals generated by the heart. As such, medical devices or sensors such as CGM's that are remote from the heart may instead estimate a heart rate using an optical sensor. However, optical sensors may use a higher amount of power to monitor a heart rate compared to systems using electrical signals.

The techniques of this disclosure include a system configured to measure an impedance in various tissues (e.g., fat, muscle, etc.) to monitor at least one of a heart rate, cardiac output, vascular tone, perfusion level, fluid status, respiration rate, respiration effort, and/or other physiological parameters that may be associated therewith. For example, the system may apply a current through tissue using one or more electrodes and measure a resulting voltage across the tissue using the set of electrodes or differentiated portions of a single electrode to determine an impedance of the tissue. In this way, a sensor device of the system (including, but not limited to, a CGM or other monitoring sensor device that may or may not include one or more electrodes) that is remote from the heart may additionally monitor a heart rate and/or other cardiovascular-related parameters (or other physiological parameters) without relying on an additional optical sensor for measuring heart rate.

The techniques of this disclosure may include a system configured to output at least one physiological parameter to be further processed. Again, the at least one physiological parameter may include, for example, at least one of a heart rate, cardiac output, vascular tone, perfusion level, fluid status, respiration rate, respiration effort, and/or other physiological parameters that may be associated therewith. For example, the system may output the at least one physiological parameter to a device (e.g., a cloud, a server, etc.) outside of the system. In some examples, the system may output (e.g., cause to output on a display) the at least one physiological parameter to healthcare personnel for management of patient's health/disease state. In this way, a sensor device of the system (including, but not limited to, a CGM or other monitoring sensor device that may or may not include one or more electrodes) that is remote from the heart may facilitate a treatment of a patient without relying on an additional optical sensor for measuring the at least one physiological parameter.

In one example, this disclosure describes a system for monitoring a patient that comprises one or more processors and a sensor device implemented in circuitry. The system is configured to measure, using the sensor device, an impedance of tissue of the patient and determine, using one or more processors, a physiological parameter comprising at least one of a heart rate, cardiac output, vascular tone, perfusion level, fluid status, respiration effort, or respiration rate of the patient based on the impedance of the tissue of the patient. The system is further configured to facilitate therapy, using the one or more processors, based on the determined physiological parameter.

In another example, this disclosure describes, a method for monitoring a patient comprising measuring, using a sensor device implemented in circuitry, an impedance of tissue of the patient and determining, using one or more processors, a physiological parameter comprising at least one of a heart rate, cardiac output, vascular tone, perfusion level, fluid status, respiration effort, or respiration rate of the patient based on the impedance of the tissue of the patient. The method further comprises facilitating therapy, using the one or more processors, based on the determined physiological parameter.

In one example, this disclosure describes, a system for therapy delivery comprising a sensor device, a patient device, an infusion set, and an insulin pump. The sensor device is implemented in circuitry and configured to measure an impedance of tissue of the patient. The patient device is implemented in circuitry and configured to determine a physiological parameter comprising at least one of a heart rate, cardiac output, vascular tone, perfusion level, fluid status, respiration effort, or respiration rate of the patient based on the impedance of the tissue of the patient and determine an amount of insulin to be provided to the patient based on the determined physiological parameter. The insulin pump is coupled to the infusion set through tubing and configured to provide the amount of insulin to the patient.

The details of one or more aspects of the disclosure are set forth in the accompanying drawings and the description below. Other features, objects, and advantages of this disclosure will be apparent from the description and drawings, and from the claims.

### BRIEF DESCRIPTION OF DRAWINGS

FIG. 1 is a block diagram illustrating an example system for delivering or guiding therapy dosage, in accordance with one or more examples described in this disclosure.

FIG. 2 is a block diagram illustrating another example system for delivering or guiding therapy dosage, in accordance with one or more examples described in this disclosure.

FIG. 3 is a block diagram illustrating another example system for delivering or guiding therapy dosage, in accordance with one or more examples described in this disclosure.

FIG. 4 is a block diagram illustrating an example of a patient device, in accordance with one or more examples described in this disclosure.

FIG. 5 is a block diagram illustrating an example of a glucose sensing device, in accordance with one or more examples described in this disclosure.

FIG. 6 is a block diagram illustrating an example of a glucose sensing device using three electrodes for sensing, in accordance with one or more examples described in this disclosure.

FIG. 7 is a block diagram illustrating an example of a glucose sensing device using four electrodes for sensing, in accordance with one or more examples described in this disclosure.

FIG. 8 is a block diagram illustrating an example of a glucose sensing using a single probe inserted into the skin of a patent, in accordance with one or more examples described in this disclosure.

FIG. 9 is a flow chart illustrating an example process for using a sensed impedance of tissue of a patient to determine a heart rate, in accordance with one or more examples described in this disclosure.

FIG. 10 is a flow chart illustrating an example process for using a sensed impedance of tissue of a patient to determine a level of tissue perfusion, in accordance with one or more examples described in this disclosure.

FIG. 11 is a flow chart illustrating an example process for using a sensed impedance of tissue of a patient to determine a respiration rate, in accordance with one or more examples described in this disclosure.

FIG. 12 is a flow chart illustrating an example process for using a sensed impedance of tissue of a patient to determine at least one physiological parameter for facilitating treatment, in accordance with one or more examples described in this disclosure.

### DETAILED DESCRIPTION

Devices, systems, and techniques for monitoring a glucose level in a patient are described in this disclosure. External or implantable medical devices may use electrodes to perform various measurements for a patient. For example, a continuous glucose monitoring (CGM) device may include three electrodes to perform glucose sensing. Medical devices, such as the CGM device, may be located far away from the heart, which may attenuate levels of electrical signals generated by the heart and sensed by the CGM device. As used herein, devices that are located far away from the heart may include, for example, around a wrist, on an upper arm, in an abdominal region, above a chest (e.g., on a neck or on a head) or another location far away from the heart. As such, medical devices that are remote from the heart may instead estimate or augment the sensing of a heart rate using an additional and/or separate optical sensor. However, optical sensors may use or require a higher amount of power to monitor a heart rate compared to systems using electrical signals. In some examples, however, techniques described here may include the electrodes and also an optical sensor configured to estimate a heart rate.

The techniques of this disclosure include a device configured to measure an impedance in various tissues (e.g., fat, muscle, etc.) to monitor a physiological parameter (e.g., a heart rate, a cardiac output, a vascular tone, a perfusion level, a fluid status, a respiration effort, or a respiration rate). For example, a CGM device may include three electrodes to perform glucose sensing. In this example, the CGM device may be configured to use some or all of the three electrodes for performing glucose sensing to also measure impedance in tissue and to estimate a physiological parameter (e.g., a heart rate, a perfusion level, or a respiration rate) using the measured impedance. For example, the CGM device may apply a current through tissue using a pair of electrodes and measure a resulting voltage across the tissue using the electrodes to determine an impedance of the tissue. In this way, a CGM device that is remote from the heart may additionally monitor, for example, a heart rate without relying on an additional optical sensor for measuring heart rate. Similarly, the CGM device may also be used to determine a perfusion level and/or a respiration rate without relying on sensor devices that are in addition to electrodes used to perform glucose sensing. In some examples, however, additional sensor devices may be used to determine a perfusion level and/or a respiration rate. These additional physiological parameters, including but not limited to heart rate, cardiac output, vascular tone, perfusion level, fluid status, respiration effort and/or respiration rate, may be indicative of a patient's response or likely response to a diabetes therapy (such as the administration of basal or bolus insulin), or otherwise be indicative and/or predictive of a patient's overall health. As such, techniques described herein may permit an impedance measurement via electrodes to be advantageously achieved within a diabetes-oriented device, thereby potentially providing an additional sensing function for a device that is already provided for diabetes therapy and potentially providing sensing that may improve diabetes therapy and that can be used for other therapies or diagnoses.

In some examples, one or more of the impedance sense electrodes could be electrodes or differentiated portions of an electrode that is typically used for glucose monitoring, (i.e. dual-purpose electrodes or differentiated portions of a single electrode for glucose and impedance). In some examples, however, the CGM device may include additional electrodes dedicated to impedance sensing or not used for glucose monitoring but still leverage other electronics, circuitry and/or software associated with the CGM to assist in impedance measurement and/or impedance characterization for the purposes of measuring, monitoring, and/or responding to particular physiological measurements. For example, the CGM device may include additional electrodes to measure voltage across tissue using a first pair of electrodes and to provide current through the tissue using a second pair of electrodes, which may improve an accuracy of the impedance measurement compared to a CGM device that only uses a single pair of electrodes or a set of three electrodes to supply the current through the tissue and to measure the voltage across the tissue. Similarly, in some instances a single probe could be used having multiple conductive zones or differentiated portions across which an impedance measurement could be taken (comprising, for example, a single probe inserted into the skin with one electrode portion disposed nearer a proximal end of the probe near the skin surface, and a second electrode portion disposed nearer distal end of the probe deeper in the patient's cutaneous layers).

While examples described herein may refer to a physiological parameter that comprises a heart rate (e.g., heart beats per minute), in addition to heart rate, or alternatively, devices described herein may be configured to measure other physiological parameters. For example, the device may determine a cardiac output based on an impedance of the tissue. As used herein, a cardiac output may refer to a volume of blood pumped by the heart (e.g., liters per minute). In some examples, the device may determine a vascular tone based on an impedance of the tissue. As used herein, a vascular tone may refer to an amount of constriction of blood vessels.

The device may determine a perfusion level based on an impedance of the tissue. As used herein, a perfusion level may refer to a rate at which blood is delivered to tissue, i.e., a blood volume flow through a given mass of tissue, body fluid (e.g., fluid status), or another perfusion level. For instance, a perfusion level may refer to a volume of blood per unit of time per unit tissue mass (e.g., m³/(s*kg)).

In some examples, the device may determine a fluid states based on an impedance of the tissue. As used herein, fluid status may refer to how much body fluid exists in a tissue, which may be used, for example, to determine whether heart failure or failed kidney has occurred in the patient. In some examples, the device may determine a respiration effort based on an impedance of the tissue. As used herein, respiration effort may refer to an amount of chest movement for a breath, which may represent an amount of air flow per minute.

In some examples, alternatively or additionally, the device may be configured to determine a respiration rate based on a time-varying impedance of the tissue. As used herein, respiration rate may refer to a rate at which breathing occurs in a patient. For instance, respiration rate may refer to a number of breaths the patient takes per each minute.

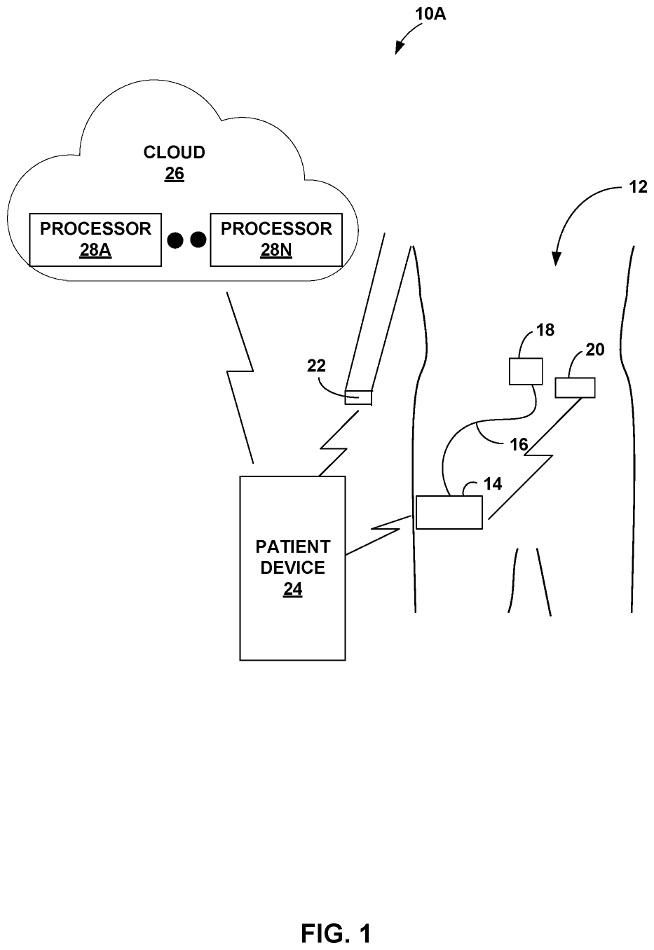

FIG. 1 is a block diagram illustrating an example system for delivering or guiding therapy dosage, in accordance with one or more examples described in this disclosure. FIG. 1 illustrates system 10A that includes patient 12, insulin pump 14, tubing 16, infusion set 18, sensor device 20, which may be a glucose sensor, wearable device 22, patient device 24, and cloud 26. Cloud 26 represents a local, wide area or global computing network including one or more processors 28A-28N (“one or more processors 28”). In some examples, the various components may determine changes to therapy based on determination of glucose level by sensor device 20, and therefore system 10A may be configured to perform glucose sensing. In some examples, system 10A may be referred to as a continuous glucose monitoring (CGM) system 10A. Additionally, system 10A may be configured for co-morbidity management (e.g., management of a heart disease and/or management of a kidney disease) in accordance with one or more techniques described herein. As described herein, system 10A may, in some examples, provide CGM and/or treatment for co-morbidity management. However, in some examples, system 10A may support another device and/or a healthcare professional that provides CGM and/or treatment for co-morbidity management.

Patient 12 may be diabetic (e.g., Type 1 diabetic or Type 2 diabetic), and therefore, the glucose level in patient 12 may be uncontrolled without delivery of supplemental insulin. For example, patient 12 may not produce sufficient insulin to control the glucose level or the amount of insulin that patient 12 produces may not be sufficient due to insulin resistance that patient 12 may have developed.

To receive the supplemental insulin, patient 12 may carry insulin pump 14 that couples to tubing 16 for delivery of insulin into patient 12. Infusion set 18 may connect to the skin of patient 12 and include a cannula to deliver insulin into patient 12. Sensor device 20 may be a continuous glucose monitoring (CGM) device that, together with patient device 24 and/or processors 28, for a CGM system. One example of sensor device 20 is Guardian Sensor 3™ by Medtronic Minimed, Inc. However, other examples of insulin pump systems may be used and the example techniques should not be considered limited to the Guardian™ Sensor 3. Sensor device 20 may be coupled to patient 12 to measure the glucose level in patient 12. For example, sensor device 20 may include one of more sensing components (e.g., electrodes) that can be percutaneously inserted into subcutaneous tissue to sense glucose levels and/or other physiological signals or conditions. Insulin pump 14, tubing 16, infusion set 18, and sensor device 20 may together form an insulin pump system. One example of the insulin pump system is the MINIMED™ 670G INSULIN PUMP SYSTEM by Medtronic Minimed, Inc. However, other examples of insulin pump systems may be used and the example techniques should not be considered limited to the MINIMED™ 670G INSULIN PUMP SYSTEM.

For example, the techniques described in this disclosure may be utilized in insulin pump systems that include wireless communication capabilities. Additionally, techniques described in this disclosure may also be utilized in other health monitoring and/or blood glucose management systems that may include, but are not limited to, CGMs in wired or wireless communication with insulin injection device 30 such as an insulin pen (including, but not limited to smart pens such as the InPen™ device from Companion Medical), or CGMs in wireless communication with diabetes or health management applications configured to be capable of running on standalone health devices or consumer electronic devices (embodied, for example, as a patient device 24) including, but not limited to, a wearable device 22 such as a smartwatch, smartphone, or other personal computing device. However, the example techniques should not be considered limited to insulin pump systems or smart insulin pens with wireless communication capabilities, and other types of communication, such as wired communication, may be possible. In another example, insulin pump 14, tubing 16, infusion set 18, and/or sensor device 20 may be contained in the same housing, including, but not limited to single-use housings such as disposable “patch pumps” having an integrated pump and glucose monitoring system in a single form factor.

Insulin pump 14 may be a relatively small device that patient 12 can place in different locations. For instance, patient 12 may clip insulin pump 14 to the waistband of pants worn by patient 12. In some examples, to be discreet, patient 12 may place insulin pump 14 in a pocket. In general, insulin pump 14 can be worn in various places and patient 12 may place insulin pump 14 in a location based on the particular clothes patient 12 is wearing.

To provide insulin, insulin pump 14 includes one or more reservoirs (e.g., two reservoirs). A reservoir may be a plastic cartridge that holds up to N units of insulin (e.g., up to 300 units of insulin) and is locked into insulin pump 14. Insulin pump 14 may be a battery powered device that is powered by replaceable and/or rechargeable batteries.

Tubing 16, sometimes called a catheter, connects on a first end to a reservoir in insulin pump 14 and connects on a second end to infusion set 18. Tubing 16 may carry the insulin from the reservoir of insulin pump 14 to patient 12. Tubing 16 may be flexible, allowing for looping or bends to minimize concern of tubing 16 becoming detached from insulin pump 14 or infusion set 18 or concern from tubing 16 breaking.

Infusion set 18 may include a thin cannula that patient 12 inserts into a layer of fat under the skin (e.g., subcutaneous connection). Infusion set 18 may rest near the stomach of patient 12. The insulin travels from the reservoir of insulin pump 14, through tubing 16, and through the cannula in infusion set 18, and into patient 12. In some examples, patient 12 may utilize an infusion set insertion device. Patient 12 may place infusion set 18 into the infusion set insertion device, and with a push of a button on the infusion set insertion device, the infusion set insertion device may insert the cannula of infusion set 18 into the layer of fat of patient 12, and infusion set 18 may rest on top of the skin of the patient with the cannula inserted into the layer of fat of patient 12.

Sensor device 20 may include a cannula that is inserted under the skin of patient 12, such as near the stomach of patient 12 or in the arm of patient 12 (e.g., subcutaneous connection). Sensor device 20 may be configured to measure, using one or more electrodes inserted under the skin and/or into tissue of the patient, the interstitial glucose level, which is the glucose found in the fluid between the cells of patient 12. Sensor device 20 may be configured to continuously or periodically sample the glucose level and rate of change of the glucose level over time.

In one or more examples, insulin pump 14 and sensor device 20, and the various components illustrated in FIG. 1, may together form a closed-loop therapy delivery system. For example, patient 12 may set a target glucose level, usually measured in units of milligrams per deciliter, on insulin pump 14. Insulin pump 14 may receive the current glucose level from sensor device 20, and in response may increase or decrease the amount of insulin delivered to patient 12. For example, if the current glucose level is higher than the target glucose level, insulin pump 14 may increase the insulin. If the current glucose level is lower than the target glucose level, insulin pump 14 may temporarily cease delivery of the insulin. Insulin pump 14 may be considered as an example of an automated insulin delivery (AID) device. Other examples of AID devices may be possible, and the techniques described in this disclosure may be applicable to other AID devices.

For example, insulin pump 14 and sensor device 20 may be configured to operate together to mimic some of the ways in which a healthy pancreas works. Insulin pump 14 may be configured to deliver basal insulin, which is a small amount of insulin released continuously throughout the day. There may be times when glucose levels increase, such as due to eating or some other activity that patient 12 undertakes. Insulin pump 14 may be configured to deliver bolus insulin on demand in association with food intake or to correct an undesirably high glucose level in the bloodstream. In one or more examples, if the sensed glucose level rises above a target level, then insulin pump 14 may increase the bolus insulin to address the increase in glucose level. Insulin pump 14 may be configured to compute basal and bolus insulin delivery, and deliver the basal and bolus insulin accordingly. For instance, insulin pump 14 may determine the amount of basal insulin to deliver continuously, and then determine the amount of bolus insulin to deliver to reduce glucose level in response to an increase in glucose level due to eating or some other event.

Accordingly, in some examples, sensor device 20 may sample glucose level and rate of change in glucose level over time. Sensor device 20 may output the glucose level to insulin pump 14 (e.g., through a wireless link connection like IEEE 802.11, Wi-Fi™, Bluetooth™ or Bluetooth Low Energy (BLE)). Insulin pump 14 may compare the glucose level to a target glucose level (e.g., as set by patient 12 or clinician), and adjust the insulin dosage based on the comparison. In some examples, sensor device 20 may also output a predicted glucose level (e.g., where glucose level is expected to be in the next 30 minutes), and insulin pump 14 may adjust insulin delivery based on the predicted glucose level. The sensor device 20 may also output glucose level or other physiological parameters measurable using the impedance determinations described herein to a variety of other wired or wirelessly-connected devices to monitor and/or treat a patient's health condition. The physiological parameters could also be sent to a medical base station, or a patient device 24 as described herein to allow a patient to self-monitor and/or augment their own wellness, treatment, or disease management activities (such as consuming food or drink, exercising, or injecting medicaments (such as insulin, for example, from a separate pen injector or smart insulin pen 30 which may be used to apply dosages in lieu of, or in addition to, the insulin pump 14).

As described above, patient 12 or a clinician may set the target glucose level on insulin pump 14 or by communication with the patient via application running on the patient device 24. There may be various ways in which patient 12 or the clinician may set the target glucose level on insulin pump 14. As one example, patient 12 or the clinician may utilize patient device 24 to communicate with insulin pump 14. Examples of patient device 24 include, but are not limited to mobile devices, such as smartwatches, smartphones, tablet computers, laptop computers, and the like. Additionally, techniques described in this disclosure may also be utilized in other health monitoring and/or blood glucose management systems different from an insulin pump 14, including, but are not limited to, CGMs in wireless communication with insulin injection pens 30 (including, but not limited to smart pens such as the InPen™ device from Companion Medical), or CGMs in wireless communication with diabetes or health management applications configured to be capable of running on standalone health devices or patient devices 24 including, but not limited to, smartwatches, smartphones, or other personal computing devices. In some examples, patient device 24 may be a special programmer or controller for insulin pump 14. Although FIG. 1 illustrates one patient device 24, in some examples, there may be a plurality of patient devices. For instance, system 10A may include a mobile device and a controller, each of which are examples of patient device 24. For ease of description only, the example techniques are described with respect to patient device 24, with the understanding that patient device 24 may be one or more patient devices.

Patient device 24 may be communicatively coupled with sensor device 20. For example, patient device 24 may be communicatively coupled with sensor device 20 via a wireless communication protocol (e.g., Wi-Fi™, IEEE 802.11, Bluetooth™, or BLE). As one example, patient device 24 may receive information from sensor device 20 through insulin pump 14, where insulin pump 14 relays the information between patient device 24 and sensor device 20. As another example, patient device 24 may receive information (e.g., glucose level or rate of change of glucose level or other physiological parameters derived from impedance measurement) directly from sensor device 20 (e.g., through a wireless link).

In one or more examples, patient device 24 may display a user interface with which patient 12 or the clinician may control insulin pump 14. For example, patient device 24 may be provided with an application or other graphical user interface for displaying a screen that allows patient 12 or the clinician to enter the target glucose level. As another example, patient device 24 may display a screen that outputs the current and/or past glucose level. In some examples, patient device 24 may output notifications to patient 12, such as notifications if the sensed glucose level is too high or too low, as well as notifications regarding any action that patient 12 needs to take. For example, if the batteries of insulin pump 14 or other medicament dispensing device, such as a smart insulin pen 30, are low on charge, then insulin pump 14 or pen 30 may output a low battery indication to patient device 24, and patient device 24 may in turn output a notification to patient 12 to replace or recharge the batteries.

Controlling insulin pump 14 through patient device 24 is one example, and should not be considered limiting. For example, insulin pump 14 may include a user interface (e.g., pushbuttons) that allows patient 12 or the clinician to set the various glucose levels of insulin pump 14. Also, in some examples, insulin pump 14 itself, or in addition to patient device 24, may be configured to output notifications to patient 12. For instance, if the sensed glucose level is too high or too low, insulin pump 14 may output an audible or haptic output. As another example, if the battery is low, then insulin pump 14 may output a low battery indication on a display of insulin pump 14.

The above describes examples ways in which insulin pump 14 may deliver insulin to patient 12 based on the current glucose levels (e.g., as measured by sensor device 20). In some cases, there may be therapeutic gains by proactively delivering insulin to patient 12, rather than reacting to when glucose levels become too high or too low.

The glucose level in patient 12 may increase due to particular user actions. As one example, the glucose level in patient 12 may increase due to patient 12 engaging in an activity like eating or exercising. In some examples, there may be therapeutic gains if it is possible to determine that patient 12 is engaging in the activity, and delivering insulin based on the determination that patient 12 is engaging in the activity.

For example, patient 12 may forget to cause insulin pump 14 to deliver insulin after eating, resulting an insulin shortfall. Alternatively, patient 12 may cause insulin pump 14 to deliver insulin after eating but may have forgotten that patient 12 previously caused insulin pump 14 to deliver insulin for the same meal event, resulting in an excessive insulin dosage. Also, in examples where sensor device 20 is utilized, insulin pump 14 may not take any action until after the glucose level is greater than a target level. By proactively determining that patient 12 is engaging in an activity, insulin pump 14 may be able to deliver insulin in such a manner that the glucose level does not rise above the target level or rises only slightly above the target level (i.e., rises by less than what the glucose level would have risen if insulin were not delivered proactively). In some cases, by proactively determining that patient 12 is engaging in an activity and delivering insulin accordingly, the glucose level of patient 12 may increase more slowly.

Although the above describes proactive determination of patient 12 eating and delivering insulin accordingly, the example techniques are not so limited. The example techniques may be utilized for proactively determining an activity that patient 12 is undertaking (e.g., eating, exercising, sleeping, driving, etc.). Insulin pump 14 may then deliver insulin based on the determination of the type of activity patient 12 is undertaking.

As illustrated in FIG. 1, patient 12 may wear wearable device 22. Examples of wearable device 22 include a smartwatch or a fitness tracker, either of which may, in some examples, be configured to be worn on a patient's wrist or arm. In one or more examples, wearable device 22 includes inertial measurement unit, such as a six-axis inertial measurement unit. The six-axis inertial measurement unit may couple a 3-axis accelerometer with a 3-axis gyroscope. Accelerometers measure linear acceleration, while gyroscopes measure rotational motion. Wearable device 22 may be configured to determine one or more movement characteristics of patient 12. Examples of the one or more movement characteristics include values relating to frequency, amplitude, trajectory, position, velocity, acceleration and/or pattern of movement instantaneously or over time. The frequency of movement of the patient's arm may refer to how many times patient 12 repeated a movement within a certain time (e.g., such as frequency of movement back and forth between two positions).

Patient 12 may wear wearable device 22 on his or her wrist. However, the example techniques are not so limited. Patient 12 may wear wearable device 22 on a finger, forearm, or bicep. In general, patient 12 may wear wearable device 22 anywhere that can be used to determine gestures indicative of eating, such as movement characteristics of the arm.

The manner in which patient 12 is moving his or her arm (i.e., the movement characteristics) may refer to the direction, angle, and orientation of the movement of the arm of patient 12, including values relating to frequency, amplitude, trajectory, position, velocity, acceleration and/or pattern of movement instantaneously or over time. As an example, if patient 12 is eating, then the arm of patient 12 will be oriented in a particular way (e.g., thumb is facing towards the body of patient 12), the angle of movement of the arm will be approximately a 90-degree movement (e.g., starting from plate to mouth), and the direction of movement of the arm will be a path that follows from plate to mouth. The forward/backward, up/down, pitch, roll, yaw measurements from wearable device 22 may be indicative of the manner in which patient 12 is moving his or her arm. Also, patient 12 may have a certain frequency at which patient 12 moves his or her arm or a pattern at which patient 12 moves his or her arm that is more indicative of eating, as compared to other activities, like smoking or vaping, where patient 12 may raise his or her arm to his or her mouth.

Although the above description describes wearable device 22 as being utilized to determine whether patient 12 is eating, wearable device 22 may be configured to detect movements of the arm of patient 12 (e.g., one or more movement characteristics), and the movement characteristics may be utilized to determine an activity undertaken by patient 12. For instance, the movement characteristics detected by wearable device 22 may indicate whether patient 12 is exercising, driving, sleeping, etc. As another example, wearable device 22 may indicate posture of patient 12, which may align with a posture for exercising, driving, sleeping, eating, etc. Another term for movement characteristics may be gesture movements.

Accordingly, wearable device 22 may be configured to detect gesture movements (i.e., movement characteristics of the arm of patient 12) and/or posture, where the gesture and/or posture may be part of various activities (e.g., eating, exercising, driving, sleeping, etc.).

In some examples, wearable device 22 may be configured to determine, based on the detected gestures (e.g., movement characteristics of the arm of patient 12) and/or posture, the particular activity patient 12 is undertaking. For example, wearable device 22 may be configured to determine whether patient 12 is eating, exercising, driving, sleeping, etc. In some examples, wearable device 22 may output information indicative of the movement characteristics of the arm of patient 12 and/or posture of patient 12 to patient device 24, and patient device 24 may be configured to determine the activity patient 12 is undertaking.

Wearable device 22 and/or patient device 24 may be programmed with information that wearable device 22 and/or patient device 24 utilize to determine the particular activity patient 12 is undertaking. For example, patient 12 may undertake various activities throughout the day where the movement characteristics of the arm of patient 12 may be similar to the movement characteristics of the arm of patient 12 for a particular activity, but patient 12 is not undertaking that activity. As one example, patient 12 yawning and cupping his or her mouth may have a similar movement as patient 12 eating. Patient 12 picking up groceries may have similar movement as patient 12 exercising. Also, in some examples, patient 12 may be undertaking a particular activity, but wearable device 22 and/or patient device 24 may fail to determine that patient 12 is undertaking the particular activity.

Accordingly, in one or more examples, wearable device 22 and/or patient device 24 may “learn” to determine whether patient 12 is undertaking a particular activity. However, the computing resources of wearable device 22 and patient device 24 may be insufficient to performing the learning needed to determine whether patient 12 is undertaking a particular activity. It may be possible for the computing resources of wearable device 26 and patient device 24 to be sufficient to perform the learning, but for ease of description only, the following is described with respect to one or more processors 28 in cloud 26.

As illustrated in FIG. 1, system 10A includes cloud 26 that includes one or more processors 28. For example, cloud 26 includes a plurality of network devices (e.g., servers), and the plurality of devices each include one or more processors. One or more processors 28 may be processors of the plurality of network devices, and may be located within a single one of the network devices, or may be distributed across two or more of the network devices. Cloud 26 represents a cloud infrastructure that supports one or more processors 28 on which applications or operations requested by one or more users run. For example, cloud 26 provides cloud computing for using one or more processors 28, to store, manage, and process data on the network devices, rather than by patient device 24 or wearable device 22. One or more processors 28 may share data or resources for performing computations, and may be part of computing servers, web servers, database servers, and the like. One or more processors 28 may be in network devices (e.g., servers) within a datacenter or may be distributed across multiple datacenters. In some cases, the datacenters may be in different geographical locations.

One or more processors 28, as well as other processing circuitry described herein, can include any one or more microprocessors, digital signal processors (DSPs), application specific integrated circuits (ASICs), field programmable gate arrays (FPGAs), or any other equivalent integrated or discrete logic circuitry, as well as any combinations of such components. The functions attributed one or more processors 28, as well as other processing circuitry described herein, herein may be embodied as hardware, firmware, software or any combination thereof.

One or more processors 28 may be implemented as fixed-function circuits, programmable circuits, or a combination thereof. Fixed-function circuits refer to circuits that provide particular functionality, and are preset on the operations that can be performed. Programmable circuits refer to circuits that can be programmed to perform various tasks, and provide flexible functionality in the operations that can be performed. For instance, programmable circuits may execute software or firmware that cause the programmable circuits to operate in the manner defined by instructions of the software or firmware. Fixed-function circuits may execute software instructions (e.g., to receive parameters or output parameters), but the types of operations that the fixed-function circuits perform are generally immutable. In some examples, the one or more of the units may be distinct circuit blocks (fixed-function or programmable), and in some examples, the one or more units may be integrated circuits. One or more processors 28 may include arithmetic logic units (ALUs), elementary function units (EFUs), digital circuits, analog circuits, and/or programmable cores, formed from programmable circuits. In examples where the operations of one or more processors 28 are performed using software executed by the programmable circuits, memory (e.g., on the servers) accessible by one or more processors 28 may store the object code of the software that one or more processors 28 receive and execute.

One example way in which one or more processors 28 may be configured to determine that patient 12 is undertaking an activity and determine therapy to deliver is described in U.S. Patent Publication No. 2020/0135320 A1. In general, one or more processors 28 may first go through an initial “learning” phase, in which one or more processors 28 receive information to determine behavior patterns of patient 12. Some of this information may be provided by patient 12. For example, patient 12 may be prompted or may himself/herself enter information into patient device 24 indicating that patient 12 is undertaking a particular activity, the length of the activity, and other such information that one or more processors 28 can utilize to predict behavior of patient 12. After the initial learning phase, one or more processors 28 may still update the behavior patterns based on more recent received information, but require fewer to no information from patient 12.

However, there may be other examples of contextual information for patient 12 such as sleep pattern, body temperature, stress level (e.g., based on pulse and respiration), heart rate, perfusion level, respiration rate, etc. In general, there may be various biometric sensors (e.g., to measure temperature, pulse/heart rate, breathing rate, etc.), which may be part of wearable device 22, part of device 20, and/or may be separate sensors. In some examples, the biometric sensors may be part of sensor device 20.

The contextual information for patient 12 may include conditional information. For example, patient 12 may eat every 3 hours, but the exact times of when patient 12 eats may be different. In some examples, the conditional information may be a determination of whether patient 12 has eaten and if a certain amount of time (e.g., 3 hours) has passed since patient 12 ate. In general, any information that establishes a pattern of behavior may be utilized to determine whether patient 12 is engaging in a particular activity.

While the above example techniques may be beneficial in patient 12 receiving insulin at the right time, this disclosure also describes example techniques to further proactively control delivery of insulin to patient 12. In accordance with the techniques of the disclosure, system 10A may be configured to measure an impedance of tissue of patient 12. For example, sensor device 20 may be configured to apply electrical current through tissue of patient 12 and, while applying the electrical current, measure an impedance of tissue of the patient. In some examples, sensor device 20 may be configured to apply a voltage at a tissue of patient 12 and, while applying the voltage, measure an impedance of tissue of the patient.

In this example, system 10A may determine a heart rate of patient 12 based on the time-varying impedance of the tissue of patient 12. For example, patient device 24 may receive an indication of the measured impedance and determine the heart rate of patient 12 based on the impedance of the tissue of patient 12. For instance, patient device 24 may determine whether the impedance of tissue of the patient corresponds to a first local maximum of a plurality of impedance values for the tissue and determine a time between the first local maximum and a second local maximum of the plurality of impedance values for the tissue. In this instance, patient device 24 may determine the heart rate using the time between the first local maximum and the second local maximum. In some examples, cloud 26 may receive an indication of the measured impedance and determine the heart rate of patient 12 based on the impedance of the tissue of patient 12.

System 10A may facilitate therapy based on the heart rate. For example, patient device 24 may determine an amount of insulin to provide based on the heart rate and may work with, e.g., instruct or control, insulin pump 14 to provide the amount of insulin to patient 12. For instance, patient device 24 may determine that patient 12 has a sensed glucose level below a glucose threshold and increase an amount of insulin to be injected into patient 12 when the heart rate is above a heart rate threshold (or outside of a range of heart rate threshold values). The heart rate threshold and/or range of heart rate threshold values may be patient-specific, pre-programed, or set by a user or administrator. In some instances, patient device 24 may apply the heart rate to a function (e.g., implemented as a look-up-table (LUT)) to determine an amount of insulin to provide to patient 12. In some examples, cloud 26 may receive an indication of the heart rate and determine amount of insulin to be injected into patient 12 based on the heart rate of patient 12.

In some examples, patient device 24 and/or cloud 26 may determine a comorbidity with diabetes based on the heart rate, such as a cardiac comorbidity with diabetes, like cardiovascular disease. For example, patient device 24 and/or cloud 26 may monitor the heart rate over a period of time to identify the presence of and/or manage one or more conditions co-occurring with diabetes. For example, system 10A may monitor a heart condition and/or a kidney condition based on the heart rate. System 10A may output (e.g., cause an output on a display) an indication of the heart rate and zero or more physiological parameters and a healthcare professional may diagnose and/or provide a treatment for a co-morbidity. For instance, patient device 24, cloud 26, or a healthcare professional may determine atrial fibrillation as a comorbidity using the indication of the heart rate and zero or more physiological parameters. Glycemic control may reduce atrial fibrillation burden and patient device 24, and/or cloud 26 may determine atrial fibrillation burden by a heart rate variability (HRV) using a Lorenz plot methodology similar to an algorithm for atrial fibrillation detection and/or burden.

In some examples, sensor device 20, patient device 24 and/or cloud 26 may facilitate a detection and management of reduced heart rate variability (HRV), which may be an output indicator of an improved cardiac condition. In some examples, sensor device 20, patient device 24 and/or cloud 26 may be configured to provide insulin control and use physiologic parameters (e.g., cardiac physiologic parameters), for example, HRV, for athletic training. For example, during the recovery period of post-athletic training, sensor device 20 may be configured to provide a faster return to “normal” glycemic levels and “normal” cardiac HRV, which may result in more effective training. In this way, sensor device 20, patient device 24 and/or cloud 26 may be configured to help to prevent “over-training syndrome.”

In this example, system 10A may determine a perfusion level (e.g., a tissue perfusion level, body fluid (e.g., fluid status, etc.) for patient 12 based on the impedance of the tissue of patient 12. For example, patient device 24 may receive an indication of the measured impedance and determine the perfusion for patient 12 based on the impedance of the tissue of patient 12. In some examples, cloud 26 may receive an indication of the measured impedance and determine the level of perfusion of patient 12 based on the impedance of the tissue of patient 12. In some examples, cloud 26 may receive an indication of the measured impedance and determine the perfusion level for patient 12 based on the impedance of the tissue of patient 12. For example, system 10A may determine impedance values of the tissue of patient 12 averaged over a period of time to determine an indication of fluid status. As heart failure and kidney disease may be co-morbidities for diabetes, and they contribute to fluid overload, the impedance values of the tissue of patient 12 averaged over a period of time may provide information for healthcare personnel to properly care for patient 12.

System 10A may facilitate therapy based on the perfusion level. For example, patient device 24 may determine an amount of insulin to provide based on the perfusion level and may work with, e.g., control or instruct, insulin pump 14 to provide the amount of insulin to patient 12. For instance, patient device 24 may determine that patient 12 has a sensed glucose level that is below a glucose threshold and increase an amount of insulin injected into patient 12 when the perfusion level is below a perfusion level threshold (or outside of a range of perfusion level threshold values). The perfusion level threshold and/or range of perfusion level threshold values may be patient-specific, pre-programed, or set by a user or administrator. In some instances, patient device 24 may apply the perfusion level to a function (e.g., implemented as a look-up-table (LUT)) to determine an amount of insulin to provide to patient 12. In some examples, patient device 24 may determine a ramp-rate for insulin titration modulated by the deviation of perfusion level from “normal.” In some examples, cloud 26 may receive an indication of the perfusion level of patient 12 and determine amount of insulin to be injected into patient 12 based on the perfusion level of patient 12.

In some examples, patient device 24 and/or cloud 26 may determine a comorbidity with diabetes based on the perfusion level. For example, patient device 24 and/or cloud 26 may monitor the perfusion over a period of time to identify the presence of one or more conditions co-occurring with diabetes. For example, a perfusion level may be used to determine and/or manage a diabetes co-morbidity with peripheral vascular disease (PVD), neuropathy, and/or heart failure.

In this example, system 10A may determine a respiration rate for patient 12 based on the impedance of the tissue of patient 12. For example, patient device 24 may receive an indication of the measured impedance and determine the respiration rate for patient 12 based on the impedance of the tissue of patient 12. In some examples, cloud 26 may receive an indication of the measured impedance and determine the respiration rate for patient 12 based on the impedance of the tissue of patient 12. For example, system 10A may apply a dual sensor approach combining impedance derived electrocardiogram (ECG) baseline shifts (e.g., baseline oscillations corresponding to respiration) and accelerometry.

System 10A may facilitate therapy based on the respiration rate. For example, patient device 24 may determine an amount of insulin to provide based on the respiration rate and may work with insulin pump 14 to provide the amount of insulin to patient 12. For instance, patient device 24 may determine that patient 12 has a sensed glucose level that is below a glucose threshold and increase an amount of insulin injected into patient 12 when the respiration rate is above a respiration rate threshold (or outside of a range of respiration rate threshold values). The respiration rate threshold and/or range of respiration rate threshold values may be patient-specific, pre-programed, or set by a user or administrator. In some instances, patient device 24 may apply the respiration rate to a function (e.g., implemented as a look-up-table (LUT)) to determine an amount of insulin to provide to patient 12. In some examples, cloud 26 may receive an indication of the respiration rate of patient 12 and determine amount of insulin to be injected into patient 12 based on the respiration rate of patient 12.

In some examples, patient device 24 and/or cloud 26 may determine a comorbidity with diabetes based on the respiration rate. For example, patient device 24 and/or cloud 26 may monitor the respiration rate over a period of time to identify the presence of one or more conditions co-occurring with diabetes. For instance, patient device 24 and/or cloud 26 may determine, based on the respiration rate, one or more of chronic obstructive pulmonary disease (COPD), emphysema, asthma, sleep apnea, and/or an inflammatory response.

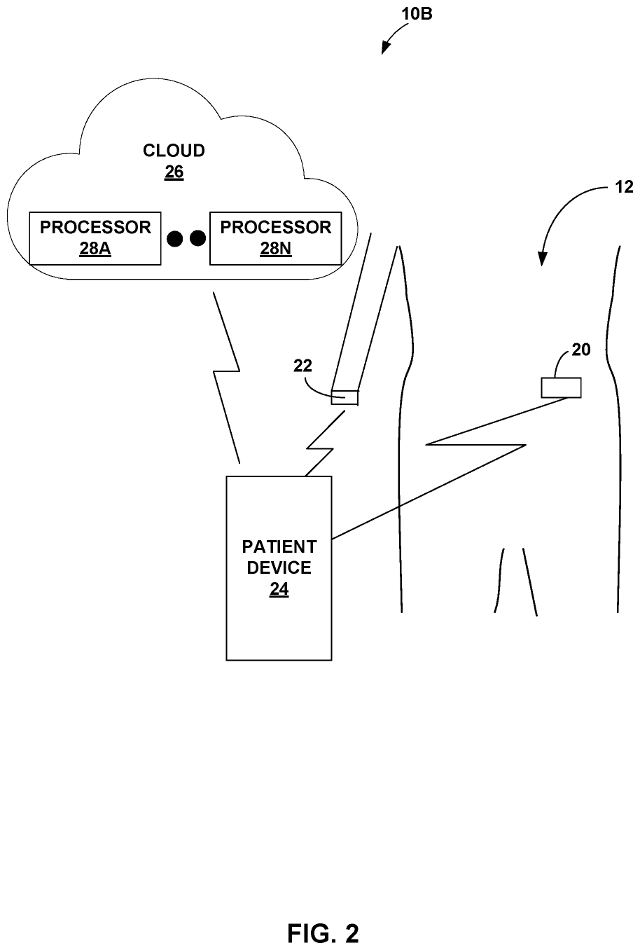

FIG. 2 is a block diagram illustrating another example system for delivering or guiding therapy dosage, in accordance with one or more examples described in this disclosure. FIG. 2 illustrates system 10B that is similar to system 10A of FIG. 1. However, in system 10B, patient 12 may not have insulin pump 14. Rather, patient 12 may utilize a manual injection device (e.g., an insulin pen or a syringe) to deliver insulin. For example, rather than insulin pump 14 automatically delivering insulin, patient 12 (or possible a caretaker of patient 12) may fill a syringe with insulin or set the dosage amount in an insulin pen and inject himself or herself. In this example, patient device 24 may be configured to output an indication of an amount of insulin to the patient for display.

System 10B may facilitate therapy based on the impedance of the tissue of patient 12. For example, patient device 24 may determine an amount of insulin to provide based on a heart rate and may output a notification on a display to inject the amount of insulin into patient 12. In some examples, patient device 24 may determine an amount of insulin to provide based on the perfusion and may output a notification on a display to inject the amount of insulin into patient 12. Patient device 24 may determine an amount of insulin to provide based on the respiration rate and may output a notification on a display to inject the amount of insulin into patient 12. For example, system 10B may use one or more of a heart rate, perfusion level, or respiration rate as confirmation sensors for glucose level to modulate insulin ramp-rate titration that is appropriate for the comorbid condition (heart failure, COPD, asthma, PVD, etc.).

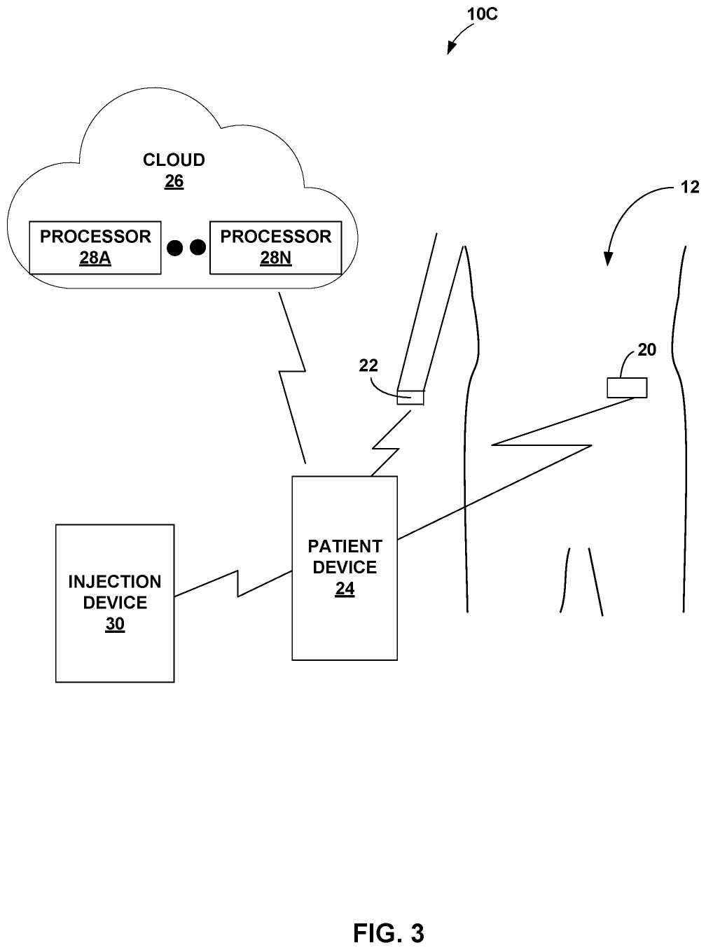

FIG. 3 is a block diagram illustrating another example system for delivering or guiding therapy dosage, in accordance with one or more examples described in this disclosure. FIG. 3 illustrates system 10C that is similar to system 10A of FIG. 1 and system 10B of FIG. 2. In system 10C, patient 12 may not have insulin pump 14. Rather, patient 12 may utilize injection device 30 to deliver insulin. For example, rather than insulin pump 14 automatically delivering insulin, patient 12 (or possible a caretaker of patient 12) may utilize injection device 30 to inject himself or herself.

Injection device 30 may be different than a syringe because injection device 30 may be a device that can communicate with patient device 24 and/or other devices in system 10C. Also, injection device 30 may include a reservoir, and based on information indicative of how much therapy dosage to deliver may be able to dose out that much insulin for delivery. For example, injection device 30 may automatically set the amount of insulin based on the information received from patient device 24. In some examples, injection device 30 may be similar to insulin pump 14, but not worn by patient 12. One example of injection device 30 is an insulin pen, sometimes embodied and equipped with electronics to render injection device 30 as a smart insulin pen capable of communicating and/or interacting with other components such as the patient device 24 or system 10C. Another example of injection device 30 may be an insulin pen with a smart cap, where the smart cap can be used to set particular doses of insulin and/or communicate with other components such as the patient device 24 or system 10C.

For example, system 10C may determine a heart rate of patient 12 based on the impedance of the tissue of patient 12. For example, patient device 24 may receive an indication of the measured impedance and determine the heart rate of patient 12 based on the impedance of the tissue of patient 12. For instance, patient device 24 may determine that patient 12 has a sensed glucose level that is below a glucose threshold and increase an amount of insulin injected into patient 12 when the heart rate is above a threshold. In some examples, cloud 26 may receive an indication of the measured impedance and determine the heart rate of patient 12 based on the impedance of the tissue of patient 12.

System 10C may facilitate therapy based on the heart rate. For example, patient device 24 may determine an amount of insulin to provide based on the heart rate and may output an instruction to cause injection device 30 to set the amount of insulin into patient 12 and to output an indication to inject the insulin into patient 12. In some examples, patient device 24 and/or cloud 26 may determine a comorbidity based on the heart rate. For example, patient device 24 and/or cloud 26 may monitor the heart rate over a period of time to identify the presence of one or more conditions co-occurring with diabetes.

In this example, system 10C may determine a perfusion (e.g., a tissue perfusion) for patient 12 based on the impedance of the tissue of patient 12. For example, patient device 24 may receive an indication of the measured impedance and determine the perfusion for patient 12 based on the impedance of the tissue of patient 12. In some examples, cloud 26 may receive an indication of the measured impedance and determine the perfusion for patient 12 based on the impedance of the tissue of patient 12.

System 10C may facilitate therapy based on the perfusion level. For example, patient device 24 may determine an amount of insulin to provide based on the perfusion level and may output an instruction to cause injection device 30 to set the amount of insulin into patient 12 and to output an indication to inject the insulin into patient 12. For instance, patient device 24 may determine that patient 12 has a sensed glucose level that is below a glucose threshold and increase an amount of insulin injected into patient 12 when the perfusion level is below a perfusion level threshold (or outside of a range of perfusion level threshold values). In some examples, patient device 24 and/or cloud 26 may determine a comorbidity based on the perfusion. For example, patient device 24 and/or cloud 26 may monitor the perfusion over a period of time to identify the presence of one or more conditions co-occurring with diabetes.

In this example, system 10C may determine a respiration rate for patient 12 based on the impedance of the tissue of patient 12. For example, patient device 24 may receive an indication of the measured impedance and determine the respiration rate for patient 12 based on the impedance of the tissue of patient 12. In some examples, cloud 26 may receive an indication of the measured impedance and determine the respiration rate for patient 12 based on the impedance of the tissue of patient 12.

System 10C may facilitate therapy based on the respiration rate. For example, patient device 24 may determine an amount of insulin to provide based on the respiration rate and may output an instruction to cause injection device 30 to set the amount of insulin into patient 12 and to output an indication to inject the insulin into patient 12. For instance, patient device 24 may determine that patient 12 has a sensed glucose level that is below a glucose threshold and increase an amount of insulin injected into patient 12 when the respiration rate is below a respiration rate threshold (or outside of a range of respiration rate threshold values). In some examples, patient device 24 and/or cloud 26 may determine a comorbidity based on the respiration rate. For example, patient device 24 and/or cloud 26 may monitor the respiration rate over a period of time to identify the presence of one or more conditions co-occurring with diabetes.

The above examples described insulin pump 14, a syringe, and injection device 30 as example ways in which to deliver insulin. In this disclosure, the term “insulin delivery device” may generally refer to any device used to deliver insulin. Examples of insulin delivery device include insulin pump 14, a syringe, and injection device 30. As described, the syringe may be a device used to inject insulin but is not necessarily capable of communicating or dosing a particular amount of insulin. Injection device 30, however, may be a device used to inject insulin that may be capable of communicating with other devices (e.g., via Wi-Fi™, IEEE 802.11, Bluetooth™, or BLE) or may be capable of dosing a particular amount of insulin. Injection device 30 may be powered (e.g., battery powered) device, and the syringe may be a device that requires no electrical power.

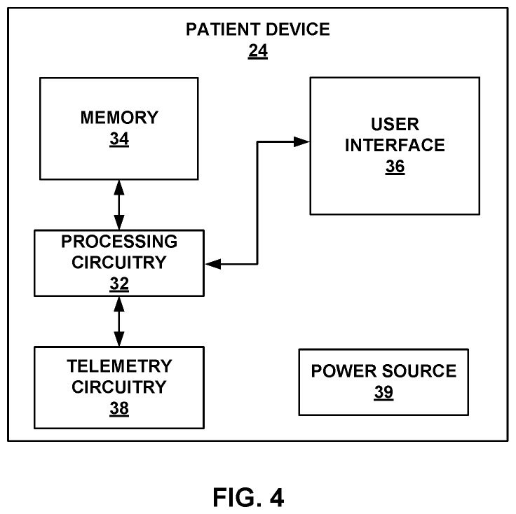

FIG. 4 is a block diagram illustrating an example of a patient device, in accordance with one or more examples described in this disclosure. While patient device 24 may generally be described as a hand-held computing device, patient device 24 may be a notebook computer, a cell phone, or a workstation, for example. In some examples, patient device 24 may be a mobile device, such as a smartphone or a tablet computer. In such examples, patient device 24 may execute an application that allows patient device 24 to perform example techniques described in this disclosure. In some examples, patient device 24 may be specialized controller for communicating with insulin pump 14.

Although the examples are described with one patient device 24, in some examples, patient device 24 may be a combination of different devices (e.g., mobile device and a controller). For instance, the mobile device may provide access to one or more processors 28 of cloud 26 through Wi-Fi™ or carrier network and the controller may provide access to insulin pump 14. In such examples, the mobile device and the controller may communicate with one another through, for example, Wi-Fi™, Bluetooth™, and/or BLE. Various combinations of a mobile device and a controller together forming patient device 24 are possible and the example techniques should not be considered limited to any one particular configuration.

As illustrated in FIG. 4, patient device 24 may include a processing circuitry 32, memory 34, user interface 36, telemetry circuitry 38, and power source 39. Memory 34 may store program instructions that, when executed by processing circuitry 32, cause processing circuitry 32 to provide the functionality ascribed to patient device 24 throughout this disclosure.

In some examples, memory 34 of patient device 24 may store a plurality of parameters, such as amounts of insulin to deliver, target glucose level, time of delivery etc. Processing circuitry 32 (e.g., through telemetry circuitry 38) may output the parameters stored in memory 34 to insulin pump 14 or injection device 30 for delivery of insulin to patient 12. In some examples, processing circuitry 32 may execute a notification application, stored in memory 34, that outputs notifications to patient 12, such as notification to take insulin, amount of insulin, and time to take the insulin, via user interface 36.

Memory 34 may include any volatile, non-volatile, fixed, removable, magnetic, optical, or electrical media, such as RAM, ROM, hard disk, removable magnetic disk, memory cards or sticks, NVRAM, EEPROM, flash memory, and the like. Processing circuitry 32 can take the form one or more microprocessors, DSPs, ASICs, FPGAs, programmable logic circuitry, or the like, and the functions attributed to processing circuitry 32 herein may be embodied as hardware, firmware, software or any combination thereof.

User interface 36 may include a button or keypad, lights, a speaker for voice commands, and a display, such as a liquid crystal (LCD). In some examples the display may be a touchscreen. As discussed in this disclosure, processing circuitry 32 may present and receive information relating to therapy via user interface 36. For example, processing circuitry 32 may receive patient input via user interface 36. The patient input may be entered, for example, by pressing a button on a keypad, entering text, or selecting an icon from a touchscreen. The patient input may be information indicative of food that patient 12 eats, such as for the initial learning phase, whether patient 12 took the insulin (e.g., through the syringe or injection device 30), and other such information.

Telemetry circuitry 38 includes any suitable hardware, firmware, software or any combination thereof for communicating with another device, such as cloud 26, insulin pump 16 or injection device 30, as applicable, wearable device 22, and sensor device 20. Telemetry circuitry 38 may receive communication with the aid of an antenna, which may be internal and/or external to patient device 24. Telemetry circuitry 38 may be configured to communicate with another computing device via wireless communication techniques, or direct communication through a wired connection. Examples of local wireless communication techniques that may be employed to facilitate communication between patient device 24 and another computing device include RF communication according to, for example, Wi-Fi™, IEEE 802.11, Bluetooth™, or BLE specification sets, infrared communication, e.g., according to an IrDA standard, or other standard or proprietary telemetry protocols. Telemetry circuitry 38 may also provide connection with carrier network for access to cloud 26. In this manner, other devices may be capable of communicating with patient device 24.

Power source 39 delivers operating power to the components of patient device 24. In some examples, power source 39 may include a battery, such as a rechargeable or non-rechargeable battery. A non-rechargeable battery may be selected to last for several years, while a rechargeable battery may be inductively charged from an external device, e.g., on a daily or weekly basis. Recharging of a rechargeable battery may be accomplished by using an alternating current (AC) outlet or through proximal inductive interaction between an external charger and an inductive charging coil within patient device 24.

In operation, telemetry circuitry 38 may receive an indication of an impedance of tissue of a patient from a sensor device (e.g., sensor device 20). In some examples, telemetry circuitry 38 may receive an indication of a single impedance value and processing circuitry 32 may store the single impedance value and respective time when the single impedance value was sensed with other impedance values of the tissue to generate a plurality of impedance values of the tissue and a time value when each respective impedance value of the plurality of impedance values was sensed.

Processing circuitry 32 may determine a heart rate of the patient based on the plurality of impedance values of the tissue sensed over a period of time. For example, processing circuitry 32 may determine a first impedance value of the plurality of impedance values corresponds to a first local maximum of the plurality of impedance values for the tissue. In this example, processing circuitry 32 may determine a time between the first local maximum and a second local maximum of the plurality of impedance values for the tissue. In this example, the time between the first local maximum and the second local maximum may represent a time between two heart beats. As such, processing circuitry 32 may determine a heart rate using the time between the first local maximum and the second local maximum. For instance, processing circuitry 32 may determine the heart rate as (1/t)*60 seconds, where t is the time between the first local maximum and the second local maximum. In some examples, telemetry circuitry 38 may output the indication of the impedance of tissue of the patient to cloud 26. In this example, cloud 26 may determine the heart rate based on the impedance of the tissue. In some examples, processing circuitry 32, with cloud 26, may determine the heart rate based on the impedance of the tissue.

In some examples, processing circuitry 32 may determine an amount of insulin to be provided to the patient based on the heart rate. For example, processing circuitry 32 may determine to increase an amount of insulin relative to an amount already set for delivery if the heart rate is outside of a range of heart rate threshold values. In some examples, processing circuitry 32 may determine a comorbidity with diabetes of the patient based on the heart rate. In some examples, cloud 26 may determine the comorbidity with diabetes based on the heart rate. In some examples, processing circuitry 32, with cloud 26, may determine the comorbidity with diabetes based on the heart rate.

Processing circuitry 32 may determine a perfusion level of the patient based on the impedance of the tissue. In some examples, telemetry circuitry 38 may output the indication of the impedance of tissue of the patient to cloud 26. For example, telemetry circuitry 38 may output an instantaneous impedance or a set of impedance samples to cloud 26. In this example, cloud 26 may determine the perfusion level based on the impedance of the tissue. In some examples, processing circuitry 32, with cloud 26, may determine the perfusion level based on the impedance of the tissue. In some examples, processing circuitry 32 may determine an amount of insulin to be provided to the patient based on the perfusion level. For example, processing circuitry 32 may determine to increase an amount of insulin to be provided to the patient based on the perfusion level being less than a perfusion threshold value. In some examples, processing circuitry 32 may determine a comorbidity with diabetes of the patient based on the perfusion level. In some examples, cloud 26 may determine the comorbidity based on the perfusion level. In some examples, processing circuitry 32, with cloud 26, may determine the comorbidity with diabetes based on the perfusion level.

Processing circuitry 32 may determine a respiration rate of the patient based on the impedance of the tissue. For example, processing circuitry 32 may determine whether the impedance of tissue of the patient corresponds to start or end of a respiration cycle. In this example, processing circuitry 32 may determine a time for the respiration cycle. Processing circuitry 32 may determine the respiration rate based on the time for the respiration cycle. In some examples, telemetry circuitry 38 may output the indication of the impedance of tissue of the patient to cloud 26. In this example, cloud 26 may determine the respiration rate based on the impedance of the tissue. In some examples, processing circuitry 32, with cloud 26, may determine the respiration rate based on the impedance of the tissue. In some examples, processing circuitry 32 may determine an amount of insulin to be provided to the patient based on the respiration rate. For example, processing circuitry 32 may determine to increase an amount of insulin to be provided to the patient based on the respiration rate being less than a respiration rate threshold value. In some examples, processing circuitry 32 may determine a comorbidity with diabetes of the patient based on the respiration rate. In some examples, cloud 26 may determine the comorbidity with diabetes based on the respiration rate. In some examples, processing circuitry 32, with cloud 26, may determine the comorbidity with diabetes based on the respiration rate.

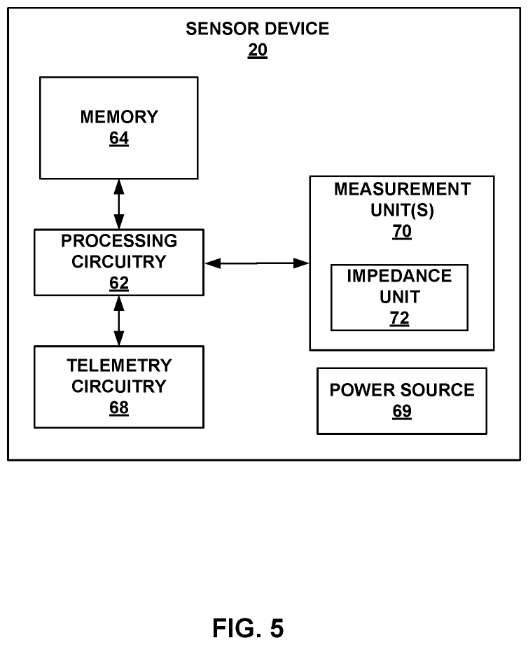

FIG. 5 is a block diagram illustrating an example of sensor device 20, in accordance with one or more examples described in this disclosure. As illustrated, device 20 includes processing circuitry 62, memory 64, telemetry circuitry 68, power source 69, and one or more sensor units 70 (also referred to herein as simply “sensor units 70”). Processing circuitry 62, memory 64, telemetry circuitry 68, and power source 69 may be similar to processing circuitry 32, memory 34, telemetry circuitry 38, and power source 39 of FIG. 3, respectively. Sensor units 70 may be configured to perform measurements of one or more physiological signals, levels or conditions a patient.

In addition, sensor units 70 may include impedance unit 72. Impedance unit 72 may be configured to measure an impedance of tissue of a patient. For example, impedance unit 72 may include a current source electrically coupled to a working electrode and a counter electrode to provide current through tissue of a patient. Again, the working electrode and a counter electrode may be configured to facilitate sensing a glucose level in patient 12. While providing the electrical current through the tissue, a voltage sensor of impedance unit 72 may detect a voltage at the tissue. For example, the voltage sensor of impedance unit 72 may detect a voltage between the working electrode and counter electrode (e.g., using 2 electrodes), between the working electrode and a reference electrode (e.g., using 3 electrodes), or between a sense working electrode and the reference electrode (e.g., using 4 electrodes). As used herein, a sense working electrode and a reference electrode may be used to transmit measurement current and not to transmit a working current. In contrast, the working electrode and counter electrode may be used to transmit the working current.

While the above example describes a constant current output, in some examples, sensor units 70 may be configured to apply a constant voltage output. For example, impedance unit 72 may be configured to measure an impedance of tissue of a patient while applying a voltage across the tissue. For example, impedance unit 72 may include a voltage source electrically coupled to a working electrode and a counter electrode to provide voltage across a tissue of a patient. Again, the working electrode and a counter electrode may be configured to facilitate sensing a glucose level in patient 12. While providing the electrical voltage through the tissue, a current sensor of impedance unit 72 may detect a current at the tissue. For example, the current sensor of impedance unit 72 may detect a voltage between the working electrode and counter electrode (e.g., using 2 electrodes), between the working electrode and a reference electrode (e.g., using 3 electrodes), or between a sense working electrode and the reference electrode (e.g., using 4 electrodes).

Telemetry circuitry 68 may output measurements of the patients. For example, telemetry circuitry 68 may output an indication of the impedance of tissue of the patient to one or more of insulin pump 14, patient device 24, wearable device 22, or processor(s) 28 in cloud 26. In some examples, processing circuitry 62 may determine a heart rate of the patient based on the impedance of the tissue. Processing circuitry 62 may determine an amount of insulin to be provided to the patient based on the heart rate. In some examples, processing circuitry 62 may determine a perfusion of the patient and/or a respiration rate of the patient based on the impedance of the tissue. Processing circuitry 62 may determine an amount of insulin to be provided to the patient based on the perfusion and/or respiration rate.

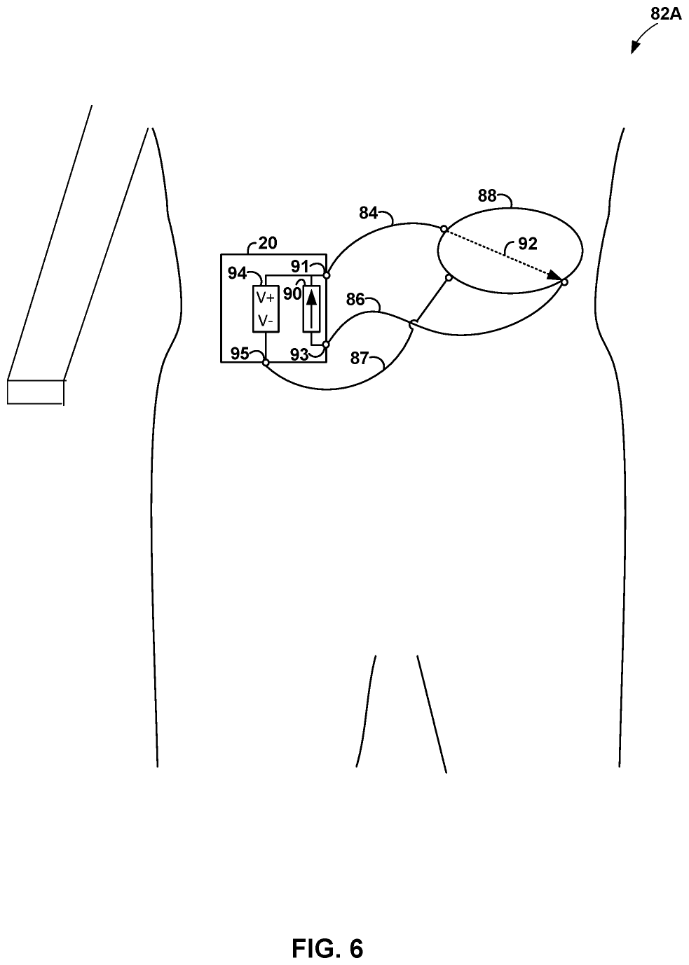

FIG. 6 is a block diagram illustrating an example of sensor device 20 using three electrodes, in accordance with one or more examples described in this disclosure. In this example, sensor device 20 has been placed on or implanted in patient 82A. In some examples, a portion of sensor device 20 may be implanted or inserted at least partially inside patient 82A, e.g., via percutaneous insertion, to place one or more sensing components within subcutaneous tissue, such as subcutaneous fatty tissue or muscle tissue. Sensor device 20 may be remote from the heart. For example, sensor device 20 may be positioned on an abdomen of patient 82A or on the back of the arm of patient 82A. Working electrode 84, counter electrode 86, and reference electrode 87 may be implanted within patient 82A. As shown in FIG. 6, sensor device 20 may include a current source 90 and voltage sensor 94. Current source 90 may be configured to generate a current 92 to flow from a first terminal 91 of sensor device 20, through working electrode 84, to tissue 88 and from tissue 88, through counter electrode 86, to a second terminal 93 of sensor device 20. Tissue 88 may refer to a perfused tissue which may include fat or muscle.

Voltage sensor 94 may be configured to sense a voltage between first terminal 91 and a third terminal 95. As shown, reference electrode 87 electrically connects third terminal 95 to tissue 88. Sensor device 20 may output an indication of the impedance to a patient device (e.g., patient device 24 of FIG. 1). For example, sensor device 20 may output, to the patient device, a logical value indicating a voltage proportional to a voltage detected between first terminal 91 and third terminal 95. In some examples, sensor device 20 may determine an impedance value based on the voltage detected between first terminal 91 and third terminal 95 and output, to the patient device, a logical value indicating the impedance value. While only an impedance measurement is discussed, with reference to FIG. 6, sensor device 20 may perform additional measurements. For example, sensor device 20 may perform measurements to determine a glucose level for patient 82A. In this way, sensor device 20 may perform glucose measurement and impedance measurements for substantially the same tissue or volume or region of tissue, using components residing in substantially the same tissue or volume or region of tissue.

While the example of FIG. 6 describes a constant current output, in some examples, sensor device 20 may be configured to apply a constant voltage output. For example, sensor device 20 may be configured to measure an impedance of tissue of a patient while applying a voltage across tissue 88. For example, sensor device 20 may include a voltage source instead of current source 90 that is electrically coupled to working electrode 84 and counter electrode 86 to provide voltage across tissue 88 of patient 82A. Again, working electrode 84 and counter electrode 86 may be configured to facilitate sensing a glucose level in patient 82A. While providing the electrical voltage through tissue 88, a current sensor may be used instead of voltage sensor 94 to detect a current at tissue 88. For example, the current sensor may detect a current flowing through working electrode 84 and reference electrode 87.

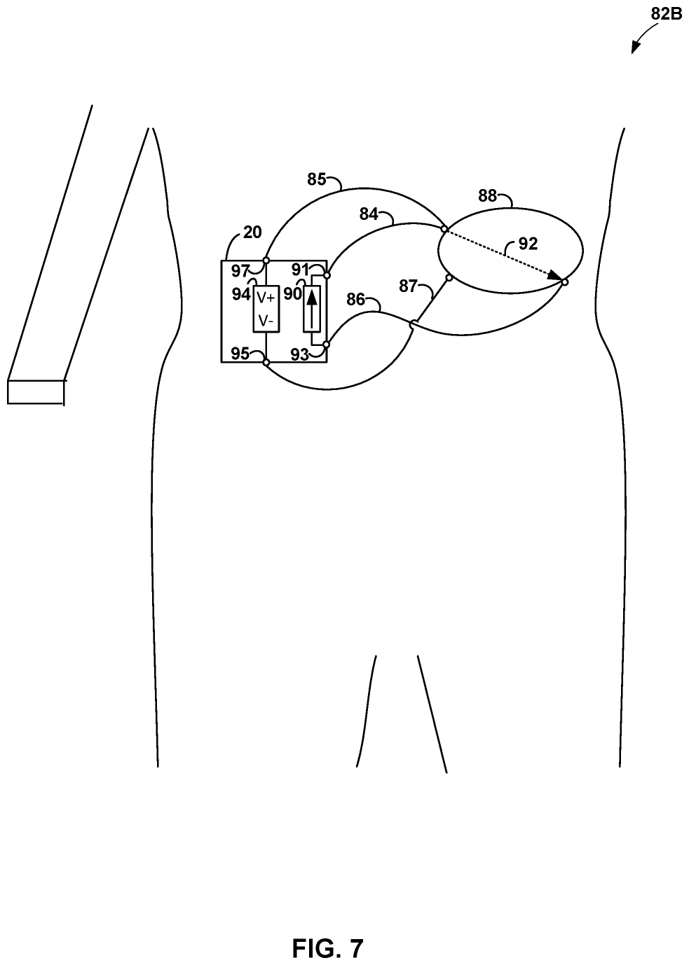

FIG. 7 is a block diagram illustrating an example of an implantable device using four electrodes, in accordance with one or more examples described in this disclosure. In this example, sensor device 20 has been placed on or implanted in patient 82B. Sensor device 20 may be remote from the heart. For example, sensor device 20 may be positioned on an abdomen of patient 82B. Working electrode 84, working sense electrode 85, counter electrode 86, and reference electrode 87 may be implanted within patient 82B. As shown in FIG. 7, sensor device 20 may include a current source 90 and voltage sensor 94. Current source 90 may be configured to generate a current 92 to flow from a first terminal 91 of sensor device 20, through a working electrode 84, to tissue 88 and from tissue 88, through a counter electrode 86, to a second terminal 93 of sensor device 20.

Voltage sensor 94 may be configured to sense a voltage between a fourth terminal 97 and a third terminal 95. As shown, a reference electrode 87 electrically connects third terminal 95 to tissue 88. Working sense electrode 85 electrically connects fourth terminal 97 to tissue 88. Sensor device 20 may output an indication of the impedance to a patient device (e.g., patient device 24 of FIG. 1). For example, sensor device 20 may output, to the patient device, a logical value indicating a voltage proportional to a voltage detected between fourth terminal 97 and third terminal 95. While only an impedance measurement is discussed, with reference to FIG. 7, sensor device 20 may perform additional measurements. For example, sensor device 20 may perform measurements to determine a glucose level for patient 82B. In this way, sensor device 20 may perform glucose measurement and impedance measurements for substantially the same tissue or volume or region of tissue, using components residing in substantially the same tissue or volume or region of tissue.

While the example of FIG. 7 describes a constant current output, in some examples, sensor device 20 may be configured to apply a constant voltage output. For example, sensor device 20 may be configured to measure an impedance of tissue of a patient while applying a voltage across tissue 88. For example, sensor device 20 may include a voltage source instead of current source 90 that is electrically coupled to working electrode 84 and counter electrode 86 to provide voltage across tissue 88 of patient 82B. Again, working electrode 84 and counter electrode 86 may be configured to facilitate sensing a glucose level in patient 82B. While providing the electrical voltage through tissue 88, a current sensor may be used instead of voltage sensor 94 to detect a current at tissue 88. For example, the current sensor may detect a current flowing through working sense electrode 85 and reference electrode 87.

The examples of FIGS. 6 and 7 comprise at least one electrode (e.g., working electrode 84) configured to only deliver current (or voltage) and not for sensing a resulting voltage (or current). However, examples may include any combination of electrodes that are configured to only deliver current (or voltage), configured to only sense a resulting voltage (or current), or configured to both delivery current (or voltage) and sense a resulting voltage (or current). For example, to measure the impedance of tissue, each electrode of sensor device 20 may be configured for delivering current to flow through the tissue, for sensing the resultant voltage, or for both delivering current to flow through the tissue and for sensing the resultant voltage.

Additionally, while the examples of FIGS. 6 and 7 comprise three or four electrodes, some examples may configure sensor device 20 to measure an impedance using only two electrodes or more than four electrodes. For example, to measure the impedance of the tissue, sensor device 20 may be configured to use two electrodes, with or without either of the electrodes implanted within the patient.

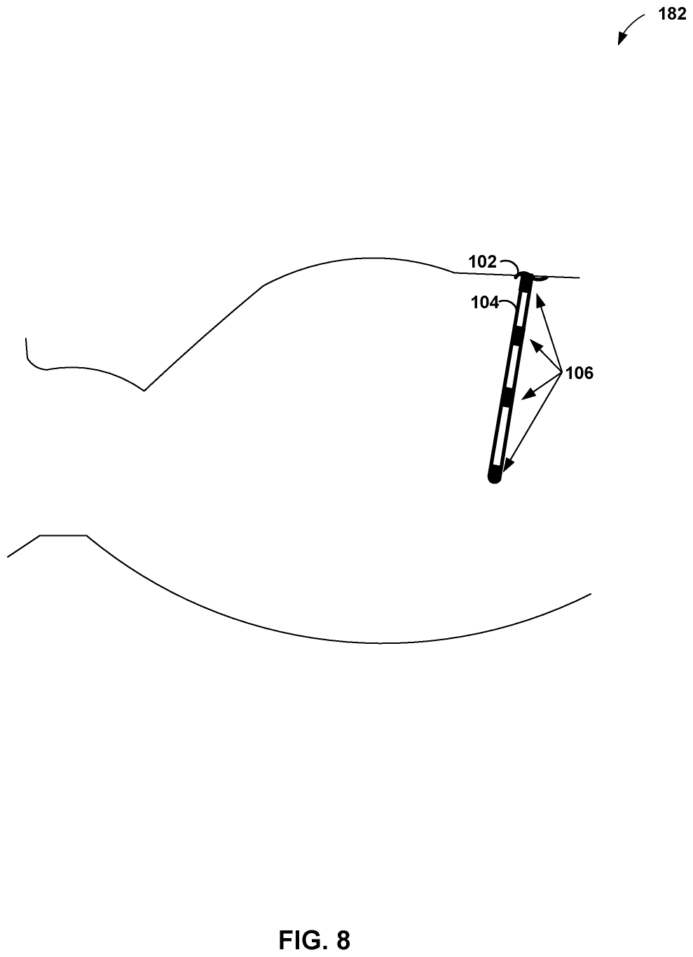

FIG. 8 is a block diagram illustrating an example of a glucose sensing using a single probe 104 inserted into the skin 102 of a patent 182, in accordance with one or more examples described in this disclosure. Single probe 104 may represent a probe plus surface electrode example. In this example, single probe 104 may be used for glucose sensing and may comprise a skin surface electrode (e.g., a conductive zone) for impedance measurement that could include a vector from a surface to single probe 104. Single probe 104 may support a measurement of impedance of tissue using three electrodes (e.g., see FIG. 6), using four electrodes (see FIG. 7), or using another number of electrodes (e.g., fewer than three electrodes or more than four electrodes). In some examples, multiple probes may be used to support a measurement of impedance of tissue using three electrodes (e.g., see FIG. 6), using four electrodes (see FIG. 7), or using another number of electrodes (e.g., fewer than three electrodes or more than four electrodes).

In some examples, single electrode 102 may comprise multiple conductive zones 106 or differentiated portions across which an impedance measurement could be taken by a sensor device (e.g., sensor device 20). For example, single probe 104 may be inserted into the skin with one electrode portion disposed nearer a proximal end of the probe near the skin surface of skin 102 of patient 182, and a second electrode portion disposed nearer distal end of the probe deeper in the patient's cutaneous layers). In some examples, a first electrode may be positioned in the tissue and a second electrode may be positioned on the surface of skin 102.

Although the example of FIG. 8 comprises a probe inserted inside a patient, some examples may include electrodes that are not inserted inside a patient. For example, one or more probes may include electrodes that are positioned on a surface of a skin of a patient. That is, all electrodes (e.g., impedance-sensing elements) of sensor device 20 may not be inserted inside the patient.

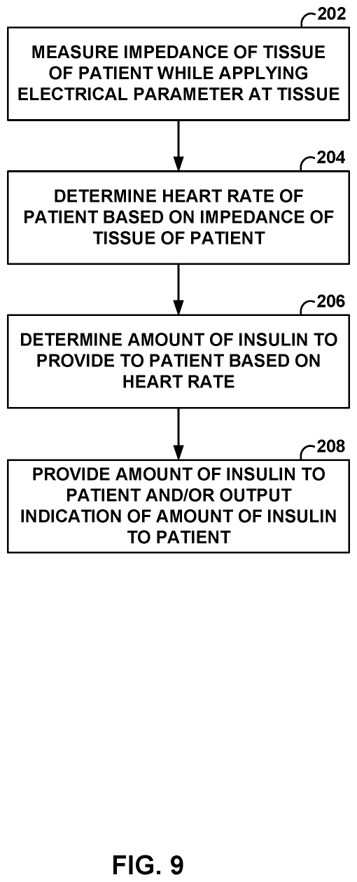

FIG. 9 is a flow chart illustrating an example process for using an impedance of tissue of a patient to determine a heart rate, in accordance with one or more examples described in this disclosure. FIG. 9 is discussed with reference to FIGS. 1-8 for example purposes only.

Sensor device 20 may measure an impedance of tissue of patient 12 while applying an electrical parameter at the tissue (202). For example, sensor device 20 may measure the impedance of tissue patient 82A using three electrodes as shown in FIG. 6. In some examples, sensor device 20 may measure the impedance of tissue patient 82B using four electrodes as shown in FIG. 7. Sensor device 20 may include a single probe (e.g., see FIG. 8) or multiple probes. The electrical parameter may comprise current or voltage. For example, sensor device 20 may apply a constant current through the tissue and measure a resulting voltage at the tissue. In some examples, sensor device 20 may apply a constant voltage across the tissue and measure a resulting current at the tissue.

System 10A may determine a heart rate of the patient based on the impedance of the tissue of the patient (204). For example, one or more of sensor device 20, wearable device 22, patient device 24, or cloud 26 determines the heart rate of the patient based on the impedance of the tissue of the patient.

System 10A may determine an amount of insulin to provide to the patient based on the heart rate (206). For example, one or more of sensor device 20, wearable device 22, patient device 24, or cloud 26 determines the amount of insulin to provide to the patient based on the heart rate. System 10A may provide the amount of insulin to the patient and/or outputs an indication of the amount of insulin to the patient (208). For example, insulin pump 14 may provide the amount of insulin to patient 12. In some examples, patient device 24 may output an indication of the amount of insulin to the patient. In some examples, injection device 30 may output an indication of the amount of insulin to the patient. In some examples, system 10A may determine a comorbidity with diabetes based on the heart rate. For example, one or more of sensor device 20, wearable device 22, patient device 24, or cloud 26 may determine the comorbidity with diabetes based on the heart rate. In some examples, system 10A may output the heart rate (e.g., at a display of patient device 24, cloud 26, or another device of system 10A) and omit steps 206 and 208.

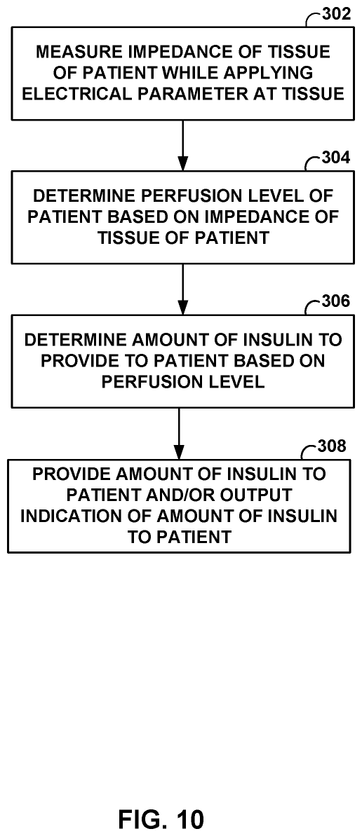

FIG. 10 is a flow chart illustrating an example process for using an impedance of tissue of a patient to determine a perfusion level, in accordance with one or more examples described in this disclosure. FIG. 10 is discussed with reference to FIGS. 1-9 for example purposes only.

Sensor device 20 may measure an impedance of tissue of patient 12 while applying an electrical parameter at the tissue (302). For example, sensor device 20 may measure the impedance of tissue patient 82A using three electrodes as shown in FIG. 6. In some examples, sensor device 20 may measure the impedance of tissue patient 82B using four electrodes as shown in FIG. 7. Sensor device 20 may include a single probe (e.g., see FIG. 8) or multiple probes. The electrical parameter may comprise current or voltage. For example, sensor device 20 may apply a constant current through the tissue and measure a resulting voltage at the tissue. In some examples, sensor device 20 may apply a constant voltage across the tissue and measure a resulting current at the tissue.

System 10A may determine a perfusion level of the patient based on the impedance of the tissue of the patient (304). For example, one or more of sensor device 20, wearable device 22, patient device 24, or cloud 26 determines a perfusion level within the tissue where the impedance is sensed based on the impedance of the tissue of the patient.

System 10A may determine an amount of insulin to provide to the patient based on the perfusion level (306). For example, one or more of sensor device 20, wearable device 22, patient device 24, or cloud 26 determines the amount of insulin to provide to the patient based on the perfusion level. System 10A may provide the amount of insulin to the patient and/or outputs an indication of the amount of insulin to the patient (308). For example, insulin pump 14 may provide the amount of insulin to patient 12. In some examples, patient device 24 may output an indication of the amount of insulin to the patient. In some examples, injection device 30 may output an indication of the amount of insulin to the patient. In some examples, system 10A may determine a comorbidity with diabetes based on the perfusion level. For example, one or more of sensor device 20, wearable device 22, patient device 24, or cloud 26 may determine the comorbidity with diabetes based on the perfusion level. In some examples, system 10A may output the perfusion level (e.g., at a display of patient device 24, cloud 26, or another device of system 10A) and omit steps 306 and 308.

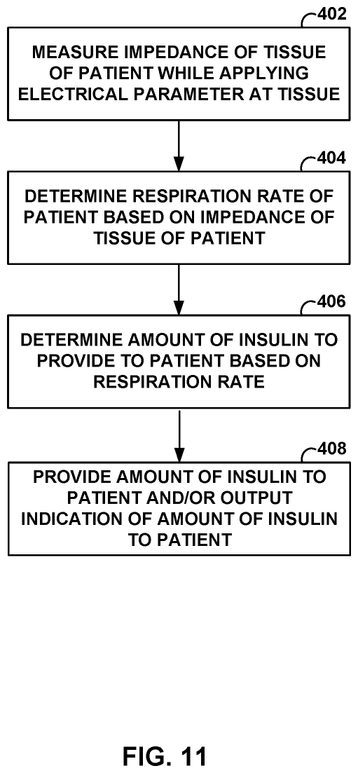

FIG. 11 is a flow chart illustrating an example process for using an impedance of tissue of a patient to determine a respiration rate, in accordance with one or more examples described in this disclosure. FIG. 11 is discussed with reference to FIGS. 1-10 for example purposes only.

Sensor device 20 may measure an impedance of tissue of patient 12 while applying an electrical parameter at the tissue (402). For example, sensor device 20 may measure the impedance of tissue patient 82A using three electrodes as shown in FIG. 6. In some examples, sensor device 20 may measure the impedance of tissue patient 82B using four electrodes as shown in FIG. 7. Sensor device 20 may include a single probe (e.g., see FIG. 8) or multiple probes. The electrical parameter may comprise current or voltage. For example, sensor device 20 may apply a constant current through the tissue and measure a resulting voltage at the tissue. In some examples, sensor device 20 may apply a constant voltage across the tissue and measure a resulting current at the tissue.

System 10A determines a respiration rate of the patient based on the impedance of the tissue of the patient (404). For example, one or more of sensor device 20, wearable device 22, patient device 24, or cloud 26 determines a respiration rate of the patient based on the impedance of the tissue of the patient.

System 10A may determine an amount of insulin to provide to the patient based on the perfusion level (406). For example, one or more of sensor device 20, wearable device 22, patient device 24, or cloud 26 determines the amount of insulin to provide to the patient based on the respiration rate. System 10A may provide the amount of insulin to the patient and/or outputs an indication of the amount of insulin to the patient (408). For example, insulin pump 14 may provide the amount of insulin to patient 12. In some examples, patient device 24 may output an indication of the amount of insulin to the patient. In some examples, injection device 30 may output an indication of the amount of insulin to the patient. In some examples, system 10A may determine a comorbidity with diabetes based on the respiration rate. For example, one or more of sensor device 20, wearable device 22, patient device 24, or cloud 26 may determine the comorbidity with diabetes based on the respiration rate. In some examples, system 10A may output the respiration rate (e.g., at a display of patient device 24, cloud 26, or another device of system 10A) and omit steps 406 and 408.

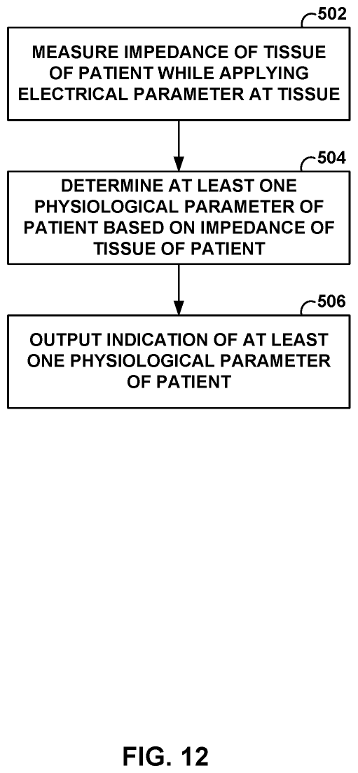

FIG. 12 is a flow chart illustrating an example process for using a sensed impedance of tissue of a patient to determine at least one physiological parameter for facilitating treatment, in accordance with one or more examples described in this disclosure. FIG. 12 is discussed with reference to FIGS. 1-11 for example purposes only. While the examples of FIGS. 9-11 are directed to physiologic parameters derived from impedance measurements for insulin control, sensor device 20 may perform glycemic control to improve cardiac conditions such as heart failure, atrial fibrillation, or other conditions.

Sensor device 20 may measure an impedance of tissue of patient 12 while applying an electrical parameter at the tissue (502). For example, sensor device 20 may measure the impedance of tissue patient 82A using three electrodes as shown in FIG. 6. In some examples, sensor device 20 may measure the impedance of tissue patient 82B using four electrodes as shown in FIG. 7. Sensor device 20 may include a single probe (e.g., see FIG. 8) or multiple probes. The electrical parameter may comprise current or voltage. For example, sensor device 20 may apply a constant current through the tissue and measure a resulting voltage at the tissue. In some examples, sensor device 20 may apply a constant voltage across the tissue and measure a resulting current at the tissue.

System 10A may determine at least one physiological parameter based on the impedance of the tissue of the patient (504). Again, the at least one physiological parameter may include, for example, at least one of a heart rate, cardiac output, vascular tone, perfusion level, fluid status, respiration rate, respiration effort, and/or other physiological parameters that may be associated therewith. For example, one or more of sensor device 20, wearable device 22, patient device 24, or cloud 26 determines at least one physiological parameter of the patient based on the impedance of the tissue of the patient.

System 10A may output an indication of the at least on physiological parameter of the patient (506). For example, system 10A may cause an output at a display of patient device 24, cloud 26, or another device of system 10A for display to a healthcare professional. In this example, the healthcare professional may diagnose and/or provide a treatment for a co-morbidity with diabetes or another disease.

In some examples, sensor device 20 may facilitate a detection and management of reduced heart rate variability (HRV), which may be an output indicator of an improved cardiac condition. In some examples, sensor device 20 may be configured to provide insulin control and use physiologic parameters (e.g., cardiac physiologic parameters), for example, HRV, for athletic training. For example, during the recovery period of post-athletic training, sensor device 20 may be configured to provide a faster return to “normal” glycemic levels and “normal” cardiac HRV, which may result in more effective training. In this way, sensor device 20 may be configured to help to prevent “over-training syndrome.”

Various aspects of the techniques may be implemented within one or more processors, including one or more microprocessors, DSPs, ASICs, FPGAs, or any other equivalent integrated or discrete logic circuitry, as well as any combinations of such components, embodied in programmers, such as physician or patient programmers, electrical stimulators, or other devices. The term “processor” or “processing circuitry” may generally refer to any of the foregoing logic circuitry, alone or in combination with other logic circuitry, or any other equivalent circuitry.

In one or more examples, the functions described in this disclosure may be implemented in hardware, software, firmware, or any combination thereof. If implemented in software, the functions may be stored on, as one or more instructions or code, a computer-readable medium and executed by a hardware-based processing unit. Computer-readable media may include computer-readable storage media forming a tangible, non-transitory medium. Instructions may be executed by one or more processors, such as one or more DSPs, ASICs, FPGAs, general purpose microprocessors, or other equivalent integrated or discrete logic circuitry. Accordingly, the term “processor,” as used herein may refer to one or more of any of the foregoing structure or any other structure suitable for implementation of the techniques described herein.

In addition, in some aspects, the functionality described herein may be provided within dedicated hardware and/or software modules. Depiction of different features as modules or units is intended to highlight different functional aspects and does not necessarily imply that such modules or units must be realized by separate hardware or software components. Rather, functionality associated with one or more modules or units may be performed by separate hardware or software components, or integrated within common or separate hardware or software components. Also, the techniques could be fully implemented in one or more circuits or logic elements. The techniques of this disclosure may be implemented in a wide variety of devices or apparatuses, including one or more processors 28 of cloud 26, one or more processors of patient device 24, one or more processors of wearable device 22, one or more processors of insulin pump 14, or some combination thereof. The one or more processors may be one or more integrated circuits (ICs), and/or discrete electrical circuitry, residing in various locations in the example systems described in this disclosure.

The one or more processors or processing circuitry utilized for example techniques described in this disclosure may be implemented as fixed-function circuits, programmable circuits, or a combination thereof. Fixed-function circuits refer to circuits that provide particular functionality, and are preset on the operations that can be performed. Programmable circuits refer to circuits that can be programmed to perform various tasks, and provide flexible functionality in the operations that can be performed. For instance, programmable circuits may execute software or firmware that cause the programmable circuits to operate in the manner defined by instructions of the software or firmware. Fixed-function circuits may execute software instructions (e.g., to receive parameters or output parameters), but the types of operations that the fixed-function circuits perform are generally immutable. In some examples, the one or more of the units may be distinct circuit blocks (fixed-function or programmable), and in some examples, the one or more units may be integrated circuits. The processors or processing circuitry may include arithmetic logic units (ALUs), elementary function units (EFUs), digital circuits, analog circuits, and/or programmable cores, formed from programmable circuits. In examples where the operations of the processors or processing circuitry are performed using software executed by the programmable circuits, memory accessible by the processors or processing circuitry may store the object code of the software that the processors or processing circuitry receive and execute.

Various aspects of the disclosure have been described. These and other aspects are within the scope of the following claims.

What is claimed is:

1. A system for monitoring a patient, the system comprising one or more processors and a sensor device implemented in circuitry, the system being configured to:
measure, using the sensor device, an impedance of tissue of the patient;
determine, using one or more processors, a physiological parameter comprising at least one of a heart rate, cardiac output, vascular tone, perfusion level, fluid status, respiration effort, or respiration rate of the patient based on the impedance of the tissue of the patient; and
facilitate therapy, using the one or more processors, based on the determined physiological parameter.

2. The system of claim 1, wherein the physiological parameter comprises heart rate.

3. The system of claim 1, wherein the sensor device comprises one or more sensing elements configured to be inserted at least partially inside the patient.

4. The system of claim 1, wherein the sensor device comprises a glucose sensor configured to perform glucose sensing for use in diabetes therapy.

5. The system of claim 4, wherein, to facilitate therapy, the system is further configured to determine a comorbidity with diabetes based on the physiological parameter.

6. The system of claim 1, wherein, to facilitate therapy, the system is further configured to:
determine, using the one or more processors, an amount of insulin to be provided to the patient for diabetes therapy based on the determined physiological parameter, the system further comprising:
an insulin pump; and
an infusion set coupled to the insulin pump through tubing, wherein the insulin pump is configured to provide the amount of insulin to the patient using the infusion set.

7. The system of claim 1, wherein the physiological parameter comprises a heart rate and wherein, to facilitate therapy, the system is further configured to determine an amount of insulin to be provided to the patient based on the heart rate, the system comprising a patient device implemented in circuitry and configured to output an indication of the amount of insulin to the patient for display.

8. The system of claim 1, wherein the physiological parameter comprises a perfusion level, and wherein the system is further configured to:
determine the perfusion level of the tissue of the patient based on the impedance of the tissue of the patient; and
facilitate therapy further based on the perfusion level.

9. The system of claim 1, wherein the physiological parameter comprises a respiration rate of the patient, wherein the system is further configured to:
determine the respiration rate based on the impedance of the tissue of the patient; and
facilitate therapy further based on the respiration rate.

10. The system of claim 1, wherein the physiological parameter comprises a heart rate, and wherein, to determine the heart rate of the patient, the system is further configured to:
determine whether the impedance of tissue of the patient corresponds to a first local maximum of a plurality of impedance values for the tissue; and
determine a time between the first local maximum and a second local maximum of the plurality of impedance values for the tissue.

11. The system of claim 1, wherein, to measure the impedance of the tissue, the sensor device is configured to use three electrodes implanted within the patient.

12. The system of claim 1, wherein, to measure the impedance, the sensor device is configured to:
generate the current to flow from a first terminal of the sensor device, through a working electrode, to the tissue and from the tissue, through a counter electrode, to a second terminal of the sensor device; and
sense a voltage between the first terminal and a third terminal, wherein a reference electrode electrically connects the third terminal to the tissue.

13. The system of claim 1, wherein, to measure the impedance of the tissue, the sensor device is configured to use four electrodes implanted within the patient.

14. The system of claim 1, wherein, to measure the impedance of the tissue, the sensor device is configured to:
generate the current to flow from a first terminal of the sensor device, through a working electrode, to the tissue and from the tissue, through a counter electrode, to a second terminal of the sensor device; and
sense a voltage between a third terminal and a fourth terminal, wherein a work sense electrode electrically connects the third terminal to the tissue and wherein a reference electrode electrically connects the fourth terminal to the tissue.

15. The system of claim 1, further comprising a patient device implemented in circuitry, communicatively coupled to the sensor device, and configured to determine the physiological parameter based on the impedance of the tissue of the patient.

16. The system of claim 1, wherein at least some of the one or more processors are within a cloud and configured to determine the physiological parameter based on the impedance of the tissue of the patient.

17. The system of claim 1, wherein, to measure the impedance of the tissue, the sensor device is configured to use a plurality of electrodes implanted within the patient, the method further comprises:
determining a glucose level for the patient using the plurality of electrodes.

18. A method for monitoring a patient, the method comprising:
measuring, using a sensor device implemented in circuitry, an impedance of tissue of the patient;
determining, using one or more processors, a physiological parameter comprising at least one of a heart rate, cardiac output, vascular tone, perfusion level, fluid status, respiration effort, or respiration rate of the patient based on the impedance of the tissue of the patient; and
facilitating therapy, using the one or more processors, based on the determined physiological parameter.

19. The method of claim 18, wherein the physiological parameter comprises heart rate.

20. A system for therapy delivery, the system comprising:
a sensor device implemented in circuitry and configured to measure an impedance of tissue of the patient;
a patient device implemented in circuitry and configured to determine a physiological parameter comprising at least one of a heart rate, cardiac output, vascular tone, perfusion level, fluid status, respiration effort, or respiration rate of the patient based on the impedance of the tissue of the patient and determine an amount of insulin to be provided to the patient based on the determined physiological parameter;
an infusion set; and
an insulin pump coupled to the infusion set through tubing and configured to provide the amount of insulin to the patient.
